(19) United States
(12) Patent Application Publication
(10) Pub. No.: US 2022/0313756 A1
(43) Pub. Date: Oct. 6, 2022

(54) METHOD FOR ALLEVIATING HELICOBACTER PYLORI-ASSOCIATED DISORDER USING CULTURE OF LACTIC ACID BACTERIAL STRAIN

(71) Applicant: GLAC BIOTECH CO., LTD., Tainan City (TW)

(72) Inventors: Hsieh-Hsun HO, Tainan City (TW); Ching-Wei CHEN, Tainan City (TW); Yi-Wei KUO, Tainan City (TW); Chi-Huei LIN, Tainan City (TW); Jui-Fen CHEN, Tainan City (TW)

(21) Appl. No.: 17/491,991

(22) Filed: Oct. 1, 2021

(30) Foreign Application Priority Data
Mar. 31, 2021 (TW) 110111886

(51) Int. Cl. A61K 35/747; C12N 1/20

(52) U.S. Cl. CPC A61K 35/747; C12N 1/20; C12R 2001/25

(57) ABSTRACT

Disclosed herein is use of a culture of at least one lactic acid bacterial strain for alleviating a Helicobacter pylori-associated disorder. The at least one lactic acid bacterial strain is selected from the group consisting of Lactobacillus rhamnosus F-1 which is deposited at the China Center for Type Culture Collection (CCTCC) under an accession number CCTCC M 2011124, Lactobacillus plantarum LPL28 which is deposited at the China General Microbiological Culture Collection Center (CGMCC) under an accession number CGMCC 17954, and Lactobacillus acidophilus TYCA06 which is deposited at the CGMCC under an accession number CGMCC 15210.

### CROSS-REFERENCE TO RELATED APPLICATION

This application claims priority of Taiwanese Invention Patent Application No. 110111886, filed on Mar. 31, 2021.

### FIELD

The present disclosure relates to a method for alleviating a Helicobacter pylori-associated disorder using at least one of cultures of three lactic acid bacterial strains.

### BACKGROUND

Helicobacter pylori is a spiral gram-negative bacterium that colonizes the human stomach. H. pylori has high urease activity and can hydrolyze urea in gastric juice to produce ammonia which elevates the pH of the microenvironment of the bacterium, and which enables the bacterium to survive in the acidic environment of the stomach. Enduring infection by H. pylori may increase the risk of suffering from gastroenteritis (such as gastritis and duodenitis), gastric ulcer, duodenal ulcer, mucosa-associated lymphoid tissue lymphoma, or even gastric adenocarcinoma.

H. pylori is typically treated with a combination of antibiotics (such as amoxicillin, clarithromycin, and metronidazole) plus a proton pump inhibitor (PPI). However, these antibiotics might cause severe side effects, and there is increasing emergence of antibiotic-resistant H. pylori that impedes the cure rate.

Probiotics are resident normal flora of the intestinal tract and believed to play important roles in regulating proper intestinal immunity and digestion by balancing intestinal microflora. These beneficial microorganisms are widely used as live microbial dietary supplements and can help restoring intestinal microfloral balance. Many species of lactic acid bacteria (LAB) are conferred with the generally recognized as safe (GRAS) status, and are widely used as probiotics.

Common LAB include Lactobacillus spp., Lactococcus spp., Pediococcus spp., Streptococcus spp., Enterococcus spp., Bifidobacterium spp., Bacillus spp., Leuconostoc spp., etc. LAB have been shown to be capable of inhibiting the growth of pathogenic bacteria in the gastrointestinal tract and alleviating lactose intolerance, and to have anti-cancer, anti-bacterial, anti-fatigue, and blood pressure lowering effects.

Previous studies demonstrated that certain strains of LAB are effective against H. pylori infection. For example, it has been reported in Asgari B. et al. (2019), Visc. Med., 36:137-143 that administration of Lactobacillus rhamnosus, Lactobacillus plantarum DSM 20174, Lactobacillus acidophilus DSM 20079, or a combination thereof has been demonstrated to have ability to fight against H. pylori infection in C57BL/6 mice.

In spite of the aforesaid, there is still a need to develop an effective way for alleviating a Helicobacter pylori-associated disorder.

### SUMMARY

The present disclosure provides a method for alleviating a Helicobacter pylori-associated disorder, which can alleviate at least one of the drawbacks of the prior art, and which includes administering to a subject in need thereof a probiotic composition.

The probiotic composition includes a culture of at least one lactic acid bacterial strain. The at least one lactic acid bacterial strain is selected from the group consisting of Lactobacillus rhamnosus F-1 which is deposited at the China Center for Type Culture Collection (CCTCC) under an accession number CCTCC M 2011124, Lactobacillus plantarum LPL28 which is deposited at the China General Microbiological Culture Collection Center (CGMCC) under an accession number CGMCC 17954, and Lactobacillus acidophilus TYCA06 which is deposited at the CGMCC under an accession number CGMCC 15210.

### BRIEF DESCRIPTION OF THE DRAWINGS

Other features and advantages of the present disclosure will become apparent in the following detailed description of the embodiments with reference to the accompanying drawings, of which:

FIG. 1 shows the effect of bacterial suspensions and suspension mixtures according to this disclosure in inhibiting the growth of Helicobacter pylori, in which the symbol “* * *” represents p<0.001 (compared with the blank control group);

FIG. 2 shows the effect of cell culture supernatants and supernatant mixtures according to this disclosure in inhibiting the growth of H. pylori, in which the symbols “* *” and “* * *” respectively represent p<0.01 and p<0.001 (compared with the blank control group);

FIG. 3 shows the effect of the bacterial suspensions and suspension mixtures according to this disclosure in inhibiting the secretion of urease from H. pylori, in which the symbols and “* *” and “* *” respectively represent p<0.05 and p<0.01 (compared with the blank control group); and

FIG. 4 shows the effect of the cell culture supernatants and supernatant mixtures according to this disclosure in inhibiting the secretion of urease from H. pylori, in which the symbols “*”, “* *”, and “* * *” respectively represent p<0.05, p<0.01, and p<0.001 (compared with the blank control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for alleviating a Helicobacter pylori-associated disorder, which includes administering to a subject in need thereof a probiotic composition.

The probiotic composition includes a culture of at least one lactic acid bacterial strain. The at least one lactic acid bacterial strain is selected from the group consisting of Lactobacillus rhamnosus F-1 which is deposited at the China Center for Type Culture Collection (CCTCC) under an accession number CCTCC M 2011124, Lactobacillus plantarum LPL28 which is deposited at the China General Microbiological Culture Collection Center (CGMCC) under an accession number CGMCC 17954, and Lactobacillus acidophilus TYCA06 which is deposited at the CGMCC under an accession number CGMCC 15210.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administering” or “administration” means introducing, providing or delivering the abovementioned pharmaceutical composition to a subject showing condition(s) or symptom(s) of a Helicobacter pylori-associated disorder by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the Helicobacter pylori-associated disorder may be selected from the group consisting of gastric ulcer, duodenal ulcer, gastric adenocarcinoma, gastroesophageal reflux, dyspepsia, gastritis (such as atrophic gastritis), pyrosis, and combinations thereof.

According to the present disclosure, the culture of the at least one lactic acid bacterial strain is prepared by culturing the abovementioned at least one lactic acid bacterial strain in a liquid or solid medium suitable for growth and/or proliferation thereof.

As used herein, the term “culturing” can be used interchangeably with other terms such as “fermentation” and “cultivation”.

According to the present disclosure, the liquid medium suitable for cultivation may include, but is not limited to, MRS (De Man, Rogosa and Sharpe) broth and MRS broth containing cysteine.

The procedures and conditions for cultivation may be adjusted according to practical requirements. In this regard, those skilled in the art may refer to journal articles, e.g., Hsieh P. S. et al. (2013), New Microbiol., 36:167-179.

In certain embodiments, cultivation may be conducted at a temperature ranging from 25° C. to 40° C. In an exemplary embodiment, cultivation is conducted at 37° C.

In certain embodiments, cultivation may be conducted for a time period ranging from 20 hours to 40 hours. In an exemplary embodiment, cultivation is conducted for 24 hours.

In certain embodiments, the culture of the at least one lactic acid bacterial strain is a liquid culture.

In certain embodiments, the liquid culture may have a total bacterial concentration ranging from 10⁶ CFU/mL to 10¹⁰ CFU/mL. In an exemplary embodiment, the liquid culture may have a total bacterial concentration of 10⁹ CFU/mL.

In certain embodiments, the liquid culture is substantially free of cells. In an exemplary embodiment, when Lactobacillus rhamnosus F-1, Lactobacillus plantarum LPL28, and Lactobacillus acidophilus TYCA06 are all applied, the volume ratio of the liquid cultures of Lactobacillus rhamnosus F-1, Lactobacillus plantarum LPL28, and Lactobacillus acidophilus TYCA06 in the probiotic composition ranges from 1:0.3:0.3 to 1:3:3.

As used herein, the term “substantially free of” means that the liquid culture lacks a significant amount of a specified component (i.e., lactic acid bacterial cells). In certain embodiments, the amount of the lactic acid bacterial cells does not have a measurable effect on the properties of the liquid culture. In other embodiments, the liquid culture is completely free of the bacterial cells.

According to the present disclosure, the liquid culture which is substantially free of cells is obtained by subjecting a culture formed after culturing the at least one lactic acid bacterial strain to a separation treatment to remove bacterial cells therefrom.

According to the present disclosure, the separation treatment may be performed using techniques well-known to those skilled in the art. Examples of the separation treatment may include, but are not limited to, filtration, centrifugation (such as multi-stage centrifugation), concentration, and combinations thereof.

In an exemplary embodiment, the liquid culture which is substantially free of cells is obtained by subjecting the culture formed after culturing the at least one lactic acid bacterial strain to a centrifugation treatment.

In certain embodiments, the culture of the at least one lactic acid bacterial strain may contain bacterial cells only. In an exemplary embodiment, when Lactobacillus rhamnosus F-1, Lactobacillus plantarum LPL28, and Lactobacillus acidophilus TYCA06 are all applied, Lactobacillus rhamnosus F-1, Lactobacillus plantarum LPL28, and Lactobacillus acidophilus TYCA06 in the probiotic composition are present in a number ratio ranging from 1:0.3:0.3 to 1:3:3.

According to the present disclosure, the culture which contains bacterial cells only is obtained by subjecting a culture formed after culturing the at least one lactic acid bacterial strain to the aforesaid separation treatment to remove a liquid portion therefrom.

According to the present disclosure, the probiotic composition may be formulated as a food product using a standard technique well known to one of ordinary skill in the art. For example, the probiotic composition may be directly added to an edible material or may be used to prepare an intermediate composition (e.g., a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the probiotic composition may be prepared in the form of a pharmaceutical composition. The pharmaceutical composition may be formulated into a suitable dosage form for oral, parenteral or topical administration using technology well known to those skilled in the art.

According to the present disclosure, the suitable dosage form for oral administration includes, but is not limited to, sterile powders, tablets, troches, lozenges, pellets, capsules, dispersible powders or granules, solutions, suspensions, emulsions, syrup, elixir, slurry, drops, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, subcutaneous injection, intradermal injection, and sublingual administration.

According to the present disclosure, the suitable dosage form for topical administration includes, but is not limited to, emulsions, gels, ointments, creams, patches, liniments, powders, aerosols, sprays, lotions, serums, pastes, foams, drops, suspensions, salves, and bandages.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the probiotic composition may further include a culture of a probiotic microorganism selected from the group consisting of Lactobacillus salivarius subsp. Salicinius AP-32 (BCRC 910437), Lactobacillus johnsonii MH-68 (BCRC 910438), and a combination thereof.

The dose and frequency of administration of the probiotic composition of the present disclosure may vary depending on the following factors: the severity of the illness or disorder to be treated, routes of administration, and age, physical condition and response of the subject to be treated. In general, the probiotic composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials:

### 1. Lactic Acid Bacterial (LAB) Strains

Lactobacillus rhamnosus F-1, Lactobacillus plantarum LPL28, and Lactobacillus acidophilus TYCA06 (which are disclosed in TW 1709374 B) have been deposited at the Bioresource Collection and Research Center (BCRC) of the Food Industry Research and Development Institute (FIRDI) (No. 331, Shih-Pin Rd., Hsinchu City 300, Taiwan), and have also been deposited at the China Center for Type Culture Collection (CCTCC) of Wuhan University, the College of Life Sciences (No. 299, Bayi Rd., Wuchang District, Wuhan City 430072, Hubei Province, China) or the China General Microbiological Culture Collection Center (CGMCC) of Chinese Academy of Sciences, the Institute of Microbiology (No. 1, West Beichen Rd., Chaoyang District, Beijing 100101, China) in accordance with the Budapest Treaty.

The relevant information regarding each of the LAB strains (including accession number and date of deposit) is listed in Table 1 below.

TABLE 1

| LAB strains | Accession number | Date of deposit |
|---|---|---|
| Lactobacillus | BCRC 910469 | Apr. 8, 2010 |
| rhamnosus F-1 | CCTCC M 2011124 | Apr. 10, 2011 |
| Lactobacillus | BCRC 910536 | Dec. 27, 2011 |
| plantarum LPL28 | CGMCC 17954 | Jun. 18, 2019 |
| Lactobacillus | BCRC 910813 | Jan. 18, 2018 |
| acidophilus TYCA06 | CGMCC 15210 | Jan. 15, 2018 |

In addition, five LAB strains isolated by the applicants were used as comparative strains, including Lactobacillus rhamnosus L-12 and Lactobacillus gasseri L-2 which were isolated from the breast milk of a healthy subject, and Lactobacillus plantarum L-305, Lactobacillus acidophilus L-7, and Lactobacillus casei L-10 which were isolated from the feces of a healthy subject.
2. Escherichia coli (BCRC 51534) and Helicobacter pylori (BCRC 17219) used in the following experiments were purchased from the BCRC of the FIRDI.

### General Procedures:

### 1. Statistical Analysis

All the experiments described below were performed in triplicate. The experimental data of all the test groups are expressed as mean±standard error of the mean (SEM), and were analyzed using two-tailed Student's t-test, so as to evaluate the differences between the groups. Statistical significance is indicated by p<0.05.

### Example 1. Evaluation for the Effect of Liquid Culture of LAB Strain According to this Disclosure Against Helicobacter pylori

A. Preparation of Bacterial Suspension of H. pylori

50 μL of a seed culture of H. pylori was mixed with 450 μL of a tryptic soy broth (TSB)(BD Difco), and the resultant mixture was then added to a tube containing a tryptic soy agar (TSA) slant supplemented with 5% sheep blood (Cat. No. 1080801-G1, Taiwan Prepared Media), followed by biphasic cultivation at 37° C. for 72 hours, so as to obtain a bacterial suspension having a bacterial concentration ranging from 5×10⁶ CFU/mL to 5×10⁷ CFU/mL.

### B. Preparation of Bacterial Suspension and Cell Culture Supernatant of LAB Strain

A respective one of the eight LAB strains described in section 1 of “General Experimental Materials” was inoculated in a MRS broth (Difco) supplemented with 0.05% (w/w) cysteine, followed by cultivation in an incubator (37° C.) for 24 hours to obtain a respective inoculum. Thereafter, the respective inoculum was inoculated in an amount of 2% (v/v) into a MRS broth, followed by cultivation in an incubator (37° C.) under an anaerobic condition overnight.

After centrifugation at 3,000 rpm and 4° C. for 10 minutes, the resultant cell pellet and cell culture supernatant were collected. The cell pellet was then washed with 0.1 M phosphate-buffered saline (PBS), followed by suspending in 0.1 M PBS, so as to obtain a bacterial suspension having a bacterial concentration of 10⁹ CFU/mL.

C. Preparation of Bacterial Suspension and Cell Culture Supernatant of E. coli

E. coli was inoculated in a nutrient broth (HIMEDIA), and was then cultivated in an incubator (37° C.) for 16 hours to obtain an inoculum. Thereafter, the inoculum was inoculated in an amount of 2% (v/v) into a nutrient broth, followed by cultivation in an incubator (37° C.) under an aerobic condition for 16 hours.

After centrifugation at 3,000 rpm and 4° C. for 10 minutes, the resultant cell pellet and cell culture supernatant were collected. The cell pellet was then washed with 0.1 M PBS, followed by suspending in 0.1 M PBS, so as to obtain a bacterial suspension having a bacterial concentration of 10⁸ CFU/mL.

D. Co-Cultivation of H. pylori with Bacterial Suspension of LAB Strain

The bacterial suspension of H. pylori prepared in section A of this example was divided into 21 groups, including a blank control group, a positive control group, a negative control group, ten experimental groups (i.e., experimental groups S1 to S3 and C1 to C7), and eight comparative groups (i.e., comparative groups S1 to S5 and C1 to C3). The volume of each group was 900 μL. Each of the bacterial suspensions of the experimental groups S1 to S3, comparative groups S1 to S5, positive control group, and negative control group was added with the respective testing agent as shown in Table 2 below. The bacterial suspension of the blank control group received no treatment.

TABLE 2

| | Testing agent |
|---|---|
| Group | (100 μL) |
| Blank control group | — |
| Positive control group | Amoxicillin (240 μL/mL) |
| Negative control group | Bacterial suspension of |
| | E. coli |
| Experimental group S1 | Bacterial suspension of |
| | Lactobacillus rhamnosus F-1 |
| Experimental group S2 | Bacterial suspension of |
| | Lactobacillus plantarum LPL28 |
| Experimental group S3 | Bacterial suspension of |
| | Lactobacillus acidophilus TYCA06 |
| Comparative group S1 | Bacterial suspension of |
| | Lactobacillus rhamnosus L-12 |
| Comparative group S2 | Bacterial suspension of |
| | Lactobacillus plantarum L-305 |
| Comparative group S3 | Bacterial suspension of |
| | Lactobacillus acidophilus L-7 |
| Comparative group S4 | Bacterial suspension of |
| | Lactobacillus gasseri L-2 |
| Comparative group S5 | Bacterial suspension of |
| | Lactobacillus casei L-10 |

In addition, the bacterial suspensions of Lactobacillus rhamnosus F-1, Lactobacillus plantarum LPL28, and Lactobacillus acidophilus TYCA06 were mixed in different volume ratios to obtain 10 suspension mixtures (i.e., suspension mixtures 1 to 10) each having a total bacterial concentration of 10⁹ CFU/mL. Each of the bacterial suspensions of the experimental groups C1 to C7 and comparative groups C1 to C3 was added with 100 μL of the respective suspension mixture as shown in Table 3 below.

TABLE 3

| | | Volume ratio of Lactobacillus |
|---|---|---|
| | | rhamnosus F-1, Lactobacillus |
| | Suspension | plantarum LPL28, and |
| Group | mixture | Lactobacillus acidophilus TYCA06 |
| Experimental | 1 | 1:0.3:0.3 |
| group C1 | | |
| Experimental | 2 | 1:0.5:0.5 |
| group C2 | | |
| Experimental | 3 | 1:1:1 |
| group C3 | | |
| Experimental | 4 | 1:1:2 |
| group C4 | | |
| Experimental | 5 | 1:1:3 |
| group C5 | | |
| Experimental | 6 | 1:2:1 |
| group C6 | | |
| Experimental | 7 | 1:3:1 |
| group C7 | | |
| Comparative | 8 | 1:0.1:0.1 |
| group C1 | | |
| Comparative | 9 | 1:10:1 |
| group C2 | | |
| Comparative | 10 | 1:1:10 |
| group C3 | | |

Thereafter, 1 mL of the resultant mixture of each group was added to a tube containing a TSA slant supplemented with 5% sheep blood, followed by biphasic cultivation in an incubator (37° C.) under a microaerobic condition for 48 hours. After centrifugation at 4,000 rpm for 10 minutes, the resultant cell pellet and supernatant were collected.

E. Co-Cultivation of H. pylori with Cell Culture Supernatant of LAB Strain

The bacterial suspension of H. pylori prepared in section A of this example was divided into 21 groups, including a blank control group, a positive control group, a negative control group, ten experimental groups (i.e., experimental groups S1 to S3 and C1 to C7), and eight comparative groups (i.e., comparative groups S1 to S5 and C1 to C3). The volume of each group was 900 μL. Each of the bacterial suspensions of the experimental groups S1 to S3, comparative groups S1 to S5, positive control group, and negative control group was added with the respective testing agent as shown in Table 4 below. The bacterial suspension of the blank control group received no treatment.

TABLE 4

| | Testing agent |
|---|---|
| Group | (100 μL) |
| Blank control group | — |
| Positive control group | Amoxicillin (240 μL/mL) |
| Negative control group | Cell culture supernatant of |
| | E. coli |
| Experimental group S1 | Cell culture supernatant of |
| | Lactobacillus rhamnosus F-1 |
| Experimental group S2 | Cell culture supernatant of |
| | Lactobacillus plantarum LPL28 |
| Experimental group S3 | Cell culture supernatant of |
| | Lactobacillus acidophilus TYCA06 |
| Comparative group S1 | Cell culture supernatant of |
| | Lactobacillus rhamnosus L-12 |
| Comparative group S2 | Cell culture supernatant of |
| | Lactobacillus plantarum L-305 |
| Comparative group S3 | Cell culture supernatant of |
| | Lactobacillus acidophilus L-7 |
| Comparative group S4 | Cell culture supernatant of |
| | Lactobacillus gasseri L-2 |
| Comparative group S5 | Cell culture supernatant of |
| | Lactobacillus casei L-10 |

In addition, the cell culture supernatants of Lactobacillus rhamnosus F-1, Lactobacillus plantarum LPL28, and Lactobacillus acidophilus TYCA06 were mixed in different volume ratios to obtain 10 supernatant mixtures (i.e., supernatant mixtures 1 to 10). Each of the bacterial suspensions of the experimental groups C1 to C7 and comparative groups C1 to C3 was added with 100 μL of the respective supernatant mixture as shown in Table 5 below.

TABLE 5

| | | Volume ratio of Lactobacillus |
|---|---|---|
| | | rhamnosus F-1, Lactobacillus |
| | Supernatant | plantarum LPL28, and |
| Group | mixture | Lactobacillus acidophilus TYCA06 |
| Experimental | 1 | 1:0.3:0.3 |
| group C1 | | |
| Experimental | 2 | 1:0.5:0.5 |
| group C2 | | |
| Experimental | 3 | 1:1:1 |
| group C3 | | |
| Experimental | 4 | 1:1:2 |
| group C4 | | |
| Experimental | 5 | 1:1:3 |
| group C5 | | |
| Experimental | 6 | 1:2:1 |
| group C6 | | |
| Experimental | 7 | 1:3:1 |
| group C7 | | |
| Comparative | 8 | 1:0.1:0.1 |
| group C1 | | |
| Comparative | 9 | 1:10:1 |
| group C2 | | |
| Comparative | 10 | 1:1:10 |
| group C3 | | |

Thereafter, 1 mL of the resultant mixture of each group was added to a tube containing a TSA slant supplemented with 5% sheep blood, followed by biphasic cultivation in an incubator (37° C.) under a microaerobic condition for 48 hours. After centrifugation at 4,000 rpm for 10 minutes, the resultant cell pellet and supernatant were collected.

### F. Determination of Bacterial Viability Rate

The cell pellet of each group obtained in sections D and E of this example was suspended in 1 mL of TSB, and 100 μL of the resultant mixture was then coated onto a tryptic soy blood agar plate using spread plate technique, followed by cultivation in an incubator (37° C.) under a microaerobic condition for 96 hours. The number of colonies of H. pylori on the tryptic soy blood agar plate of each group was counted.

The bacterial viability rate (%) was calculated using the following Equation (I):

A=(B/C)×100 (I)

where A=bacterial viability rate (%)
B=total bacterial count of respective group
C=total bacterial count of the blank control group

The data thus obtained were analyzed according to the method described in section 1 of “General Procedures”.

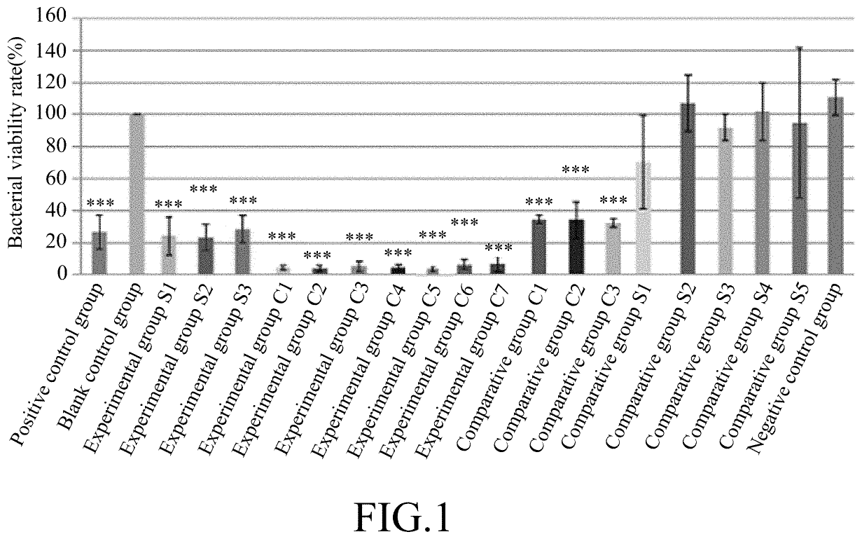

FIG. 1 shows the bacterial viability rate determined in each group after co-cultivation of H. pylori with a respective one of different bacterial suspensions or suspension mixtures. As shown in FIG. 1, the bacterial viability rates determined in the experimental groups S1 to S3 were each lower than those determined in the comparative groups S1 to S5, the negative control group, and the blank control group. In addition, the bacterial viability rates determined in the experimental groups C1 to C7 were each lower than those determined in the comparative groups C1 to C3, the negative control group, and the blank control group. In particular, the bacterial viability rates determined in the experimental groups C1 to C7 were each lower than those determined in the experimental groups S1 to S3 and the positive control group.

The aforesaid result suggests that the bacterial suspension of any one of Lactobacillus rhamnosus F-1, Lactobacillus plantarum LPL28, and Lactobacillus acidophilus TYCA06 is effective in inhibiting the growth of H. pylori, and these three LAB strains, when used in combination to prepare a suspension mixture, can synergistically exhibit a further improved efficacy.

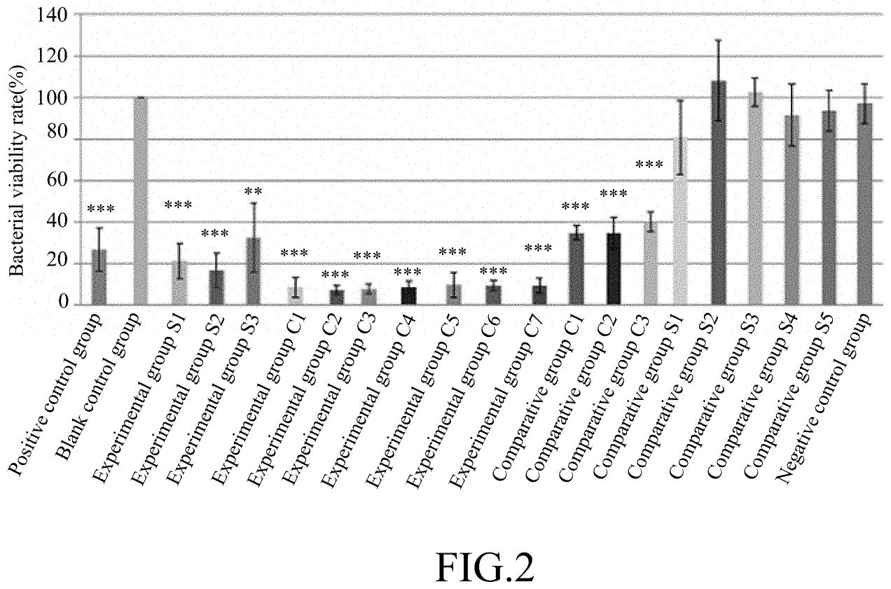

FIG. 2 shows the bacterial viability rate determined in each group after co-cultivation of H. pylori with a respective one of different cell culture supernatants or supernatant mixtures. As shown in FIG. 2, the bacterial viability rates determined in the experimental groups S1 to S3 were each lower than those determined in the comparative groups S1 to S5, the negative control group, and the blank control group. In addition, the bacterial viability rates determined in the experimental groups C1 to C7 were each lower than those determined in the comparative groups C1 to C3, the negative control group, and the blank control group. In particular, the bacterial viability rates determined in the experimental groups C1 to C7 were each lower than those determined in the experimental groups S1 to S3 and the positive control group.

This result suggests that the cell culture supernatant of any one of Lactobacillus rhamnosus F-1, Lactobacillus plantarum LPL28, and Lactobacillus acidophilus TYCA06 is effective in inhibiting the growth of H. pylori, and these three LAB strains, when used in combination to prepare a supernatant mixture, can synergistically exhibit a further improved efficacy.

### G. Determination of Urease Activity

10 μL of the supernatant of the respective group obtained in sections D and E of this example was mixed with 300 μL of an urease reaction buffer (PBS containing 20% urea and 0.012% phenol red, and having a pH of 6.5), followed by being left standing for reaction to proceed at 37° C. for 1 hour. Thereafter, the resultant mixture was subjected to determination of absorbance at a wavelength of 550 nm (OD₅₅₀) by an ELISA reader (MQX200, BioTek).

The data thus obtained were analyzed according to the method described in section 1 of “General Procedures”.

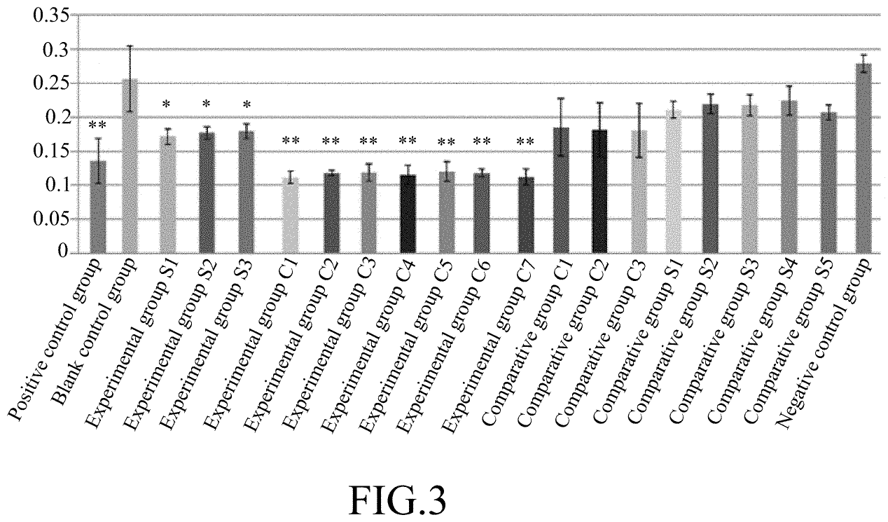

FIG. 3 shows the OD₅₅₀ value determined in each group after co-cultivation of H. pylori with a respective one of different bacterial suspensions or suspension mixtures. As shown in FIG. 3, the OD₅₅₀ values determined in the experimental groups S1 to S3 were each lower than those determined in the comparative groups S1 to S5, the negative control group, and the blank control group. In addition, the OD₅₅₀ values determined in the experimental groups C1 to C7 were each lower than those determined in the comparative groups C1 to C3, the negative control group, and the blank control group. In particular, the OD₅₅₀ values determined in the experimental groups C1 to C7 were each lower than those determined in the experimental groups S1 to S3.

This result suggests that the bacterial suspension of any one of Lactobacillus rhamnosus F-1, Lactobacillus plantarum LPL28, and Lactobacillus acidophilus TYCA06 is capable of effectively inhibiting the secretion of urease from H. pylori, and these three LAB strains, when used in combination to prepare a suspension mixture, can synergistically exhibit a further improved efficacy.

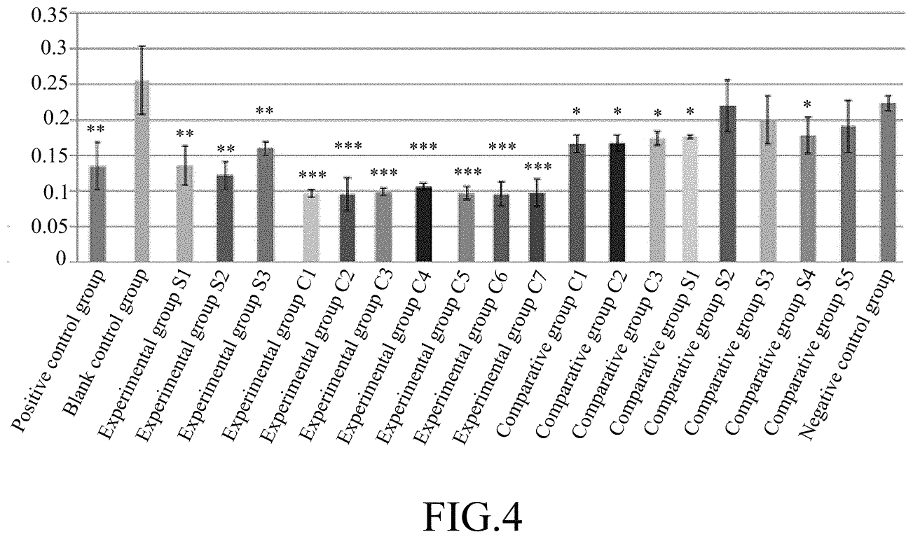

FIG. 4 shows the OD₅₅₀ value determined in each group after co-cultivation of H. pylori with a respective one of different cell culture supernatants or supernatant mixtures. As shown in FIG. 4, the OD₅₅₀ values determined in the experimental groups S1 to S3 were each lower than those determined in the comparative groups S1 to S5, the negative control group, and the blank control group. In addition, the OD₅₅₀ values determined in the experimental groups C1 to C7 were each lower than those determined in the comparative groups C1 to C3, the negative control group, and the blank control group. In particular, the OD₅₅₀ values determined in the experimental groups C1 to C7 were each lower than those determined in the experimental groups S1 to S3 and the positive control group.

This result suggests that the cell culture supernatant of any one of Lactobacillus rhamnosus F-1, Lactobacillus plantarum LPL28, and Lactobacillus acidophilus TYCA06 is capable of effectively inhibiting the secretion of urease from H. pylori, and these three LAB strains, when used in combination to prepare a supernatant mixture, can synergistically exhibit a further improved efficacy.

Summarizing the above test results, it is clear that the abovementioned bacterial suspension or cell culture supernatant of at least one lactic acid bacterial strain of this disclosure (i.e. Lactobacillus rhamnosus F-1, Lactobacillus plantarum LPL28, and Lactobacillus acidophilus TYCA06) can act effectively against H. pylori infection, and hence can alleviate a Helicobacter pylori-associated disorder.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for alleviating a Helicobacter pylori-associated disorder, comprising administering to a subject in need thereof a probiotic composition including a culture of at least one lactic acid bacterial strain, the at least one lactic acid bacterial strain being selected from the group consisting of Lactobacillus rhamnosus F-1 which is deposited at the China Center for Type Culture Collection (CCTCC) under an accession number CCTCC M 2011124, Lactobacillus plantarum LPL28 which is deposited at the China General Microbiological Culture Collection Center (CGMCC) under an accession number CGMCC 17954, and Lactobacillus acidophilus TYCA06 which is deposited at the CGMCC under an accession number CGMCC 15210.

2. The method as claimed in claim 1, wherein the Helicobacter pylori-associated disorder is selected from the group consisting of gastric ulcer, duodenal ulcer, gastric adenocarcinoma, gastroesophageal reflux, dyspepsia, gastritis, pyrosis, and combinations thereof.

3. The method as claimed in claim 1, wherein the culture is a liquid culture.

4. The method as claimed in claim 3, wherein the liquid culture is substantially free of cells.

5. The method as claimed in claim 1, wherein the culture contains bacterial cells only.

6. The method as claimed in claim 1, wherein the probiotic composition is formulated as a food product.

7. The method as claimed in claim 1, wherein the probiotic composition is formulated as a pharmaceutical composition.

8. The method as claimed in claim 7, wherein the pharmaceutical composition is in a dosage form selected from the group consisting of an oral dosage form, a parenteral dosage form, and a topical dosage form.

9. The method as claimed in claim 1, wherein the probiotic composition contains the cultures of Lactobacillus rhamnosus F-1, Lactobacillus plantarum LPL28, and Lactobacillus acidophilus TYCA06.
